(19) United States
(12) Patent Application Publication
(10) Pub. No.: US 2006/0258840 A1
(43) Pub. Date: Nov. 16, 2006

(54) Peroxisome proliferator-activated receptor

(76) Inventors: Ralph T. Mosley, Roselle, NJ (US); Brian Michael McKeever, Lake Ronkonkoma, NY (US); Joel P. Berger, Hoboken, NJ (US)

Correspondence Address: MERCK AND CO., INC, P O BOX 2000, RAHWAY, NJ, 07065-0907, US

(21) Appl. No.: 10/541,892

(22) PCT Filed: Jan. 16, 2004
(86) PCT No.: PCT/US04/01221

§ 371 Date: Jul. 8, 2005

Related U.S. Application Data
(60) Provisional application No. 60/441,836, filed on Jan. 22, 2003.

(51) Int. Cl. C07K 14/705; G01N 33/567; C07H 21/04; C12P 21/06

(52) U.S. Cl. 530/350; 536/23.5; 435/69.1; 435/320.1; 435/325; 435/7.2

(57) ABSTRACT

The present invention features mutated forms of PPAR ligand binding domain polypeptides that: (1) bind a partial PPAR agonist; and (2) is bound or activated by a full PPAR agonist to a lesser extent than the wild-type receptor. The mutated ligand binding domain contains an amino acid sequence wherein one or more interactions that preferentially (preferably solely) occurs between a full PPAR agonist and the AF-2 domain of a wild-type PPAR are modified. Preferably, the mutated ligand binding domain is selectively bound or activated by a partial PPAR agonist.

### CROSS-REFERENCE TO RELATED APPLICATIONS

The present application claims the benefit of U.S. Provisional Application No. 60/441,836, filed Jan. 22, 2003, hereby incorporated by reference herein.

### BACKGROUND OF THE INVENTION

The references cited throughout the present application are not admitted to be prior art to the claimed invention.

Nuclear receptors act as ligand-inducible transcription factors that regulate target gene expression. Regulation of target gene expression is mediated by complexes involving the nuclear receptor, agonist or antagonist ligands, and one or more coregulators. Depending on the nuclear receptor, the receptor may be present in the complex as a monomer, homodimer, or heterodimer. (Aranda et al., Physiological Reviews 81:1269-1304, 2001.)

Different nuclear receptors respond to different ligands and regulate different genes. Examples of nuclear receptors include thyroid hormone receptor, retinoic acid receptor, vitamin D receptor, peroxisome proliferator-activated receptors, pregnane X receptor, constitutive androstane receptor, liver X receptor, farnesoid X receptor, reverse ErbA, retinoid Z receptor/retinoic acid-related orphan receptor, ubiquitous receptor, retinoid X receptor, chicken ovalbumin upstream promoter transcription factor, hepatocyte nuclear factor 4, tailles-related receptor, photoreceptor-specific nuclear receptor, testis receptor, glucocorticoid receptor, androgen receptor, progesterone receptor, estrogen receptor, estrogen-related receptor, NGF-induced clone B, steroidogenic factor 1, fushi tarazu factor 1, germ cell nuclear factor, and dosage-sensitive sex reversal. (Aranda et al., Physiological Reviews 81:1269-1304, 2001.)

Nuclear receptors exhibit a modular structure with different regions corresponding to autonomous functional domains that can be interchanged between related receptors. (Aranda et al., Physiological Reviews 81:1269-1304, 2001.) A typical nuclear receptor comprises the following regions: (A/B) a variable amino terminal region containing the ligand independent AF-1 domain; (C) a conserved DNA binding domain; (D) a variable linker region; and (E) a ligand binding domain region containing the ligand-dependent AF-2 core transactivation domain. (Aranda et al., Physiological Reviews 81:1269-1304, 2001.)

An important subfamily of nuclear receptors are peroxisome proliferator activated receptors (PPAR's). The PPAR subfamily of nuclear receptors includes PPARα, PPARγ, and PPARδ (also known as PPARβ), and these receptors function as heterodimers with the retinoid X receptor (RXR). Fatty acids and eicosanoids have been identified as naturally occurring PPAR ligands. (Berger et al., Annu. Rev. Med. 53:409-435, 2002, Berger et al., Diabetes Technology & Therapeutics 4:163-174, 2002.)

Agonist or partial-agonist binding to a PPAR induces stabilization of the structure as well as a change in conformation that creates a binding cleft resulting in recruitment of transcriptional coactivators. Examples of PPAR coactivators include CBP/p300, the steroid receptor coactivator (SRC-1), members of the DRIP/TRAP complex, PGC-1, RIP140, and ARA70. The active PPAR complex is bound to a specific DNA response element mediating the rate of initiation of gene transcription. (Berger et al., Annu. Rev. Med. 53:409-435, 2002, Berger et al., Diabetes Technology & Therapeutics 4:163-174, 2002.)

Different synthetic compounds modulating a PPAR activity have been identified. (See, e.g., Berger et al., Annu. Rev. Med. 53:409-435, 2002, Berger et al., Diabetes Technology & Therapeutics 4:163-174, 2002, Acton et al. International Publication Number WO 02/08188, published Jan. 31, 2002, Berger et al., International Publication Number WO 01/30343, published May 3, 2001, Cobb et al., International Publication Number WO 01/17944, published Mar. 15, 2001.)

Partial agonists (or antagonists), also known as “selective modulators” for PPAR's have been strongly implicated as having preferred biological properties (Berger et al., International Publication Number WO 01/30343, published May 3, 2001, Moller, Nature 414:821-827, 2001, Berger et al., Annu. Rev. Med. 53:409-435, 2002). These may include the retention of selected responses which confer efficacy whereas selected responses that result in toxicity may be diminished.

### SUMMARY OF THE INVENTION

The present invention features mutated forms of PPAR ligand binding domain polypeptides that: (1) bind a partial PPAR agonist; and (2) is bound or activated by a full PPAR agonist to a lesser extent than the wild-type receptor. The mutated ligand binding domain contains an amino acid sequence wherein one or more interactions that preferentially (preferably solely) occurs between a full PPAR agonist and the AF-2 domain of a wild-type PPAR are modified. Preferably, the mutated ligand binding domain is selectively bound or activated by a partial PPAR agonist.

Selective binding or activation by a partial PPAR agonist is in comparison to activation by a full PPAR agonist. A full PPAR agonist is either a potent natural ligand or has the same type of interactions with PPAR AF-2 domain amino acids as a potent natural ligand. In contrast, a partial agonist has a significantly diminished interaction with one or more amino acids that are important for full agonist binding or activation.

A “partial PPAR agonist” can bind to a wild-type PPAR and cause detectable receptor activity, where the produced activity is less than the activity caused by a full ligand. Differences between partial and full agonist produced activity can be the type or degree of activity.

Depending upon the extent of activation caused by a partial PPAR agonist, the partial agonist can be used as an agonist or an antagonist. A partial agonist can be used in an antagonist manner, for example, by competing and diluting the effect of a naturally occurring agonist.

The ability of a mutated PPAR ligand binding domain to selectively bind a partial agonist indicates: (1) a partial agonist can bind to the mutated ligand binding domain at a comparable or greater level than it binds to the wild-type protein; and (2) a full agonist binds to the mutated ligand binding domain to a lesser extent than to the wild-type protein at a given concentration, or binds to the wild-type protein to a comparable extent, but only at a higher concentration.

The ability of a mutated PPAR ligand binding domain to be selectively activated by a partial agonist indicates: (1) a partial agonist can produce a comparable or greater response in a PPAR containing the mutated ligand binding domain than in the wild-type protein; and (2) a full agonist produces a lesser response in a PPAR containing the mutated ligand binding domain than in the wild-type protein at a given concentration, or produces a response comparable to that in the wild-type protein, but only at a higher concentration.

Reference to a “mutated” PPAR ligand binding domain indicates a different amino acid sequence than a wild-type PPAR ligand domain. Reference to “mutated” does not indicate the manner in which the “mutated” domain was produced. A “mutated” PPAR ligand binding domain can be obtained by different methods including those involving introducing a mutation into a PPAR ligand binding domain encoding nucleotide sequence, step-wise chemical synthesis of a PPAR encoding nucleotide sequence to express a “mutated” ligand binding domain, and chemically synthesizing a particular PPAR ligand binding domain amino acid sequence.

Thus, a first aspect of the present invention features a mutated PPAR ligand binding domain polypeptide. The polypeptide comprises the amino acid sequence of a mutated PPAR ligand binding domain, wherein the mutated PPAR ligand binding domain is:

(a) bound by a partial PPAR agonist; and

(b) bound or activated by a full PPAR agonist to a lesser extent than the wild-type receptor.

Activation of a mutated PPAR ligand binding domain polypeptide can be, for example, a change in conformation that would allow recruitment or binding of coactivator proteins.

Unless particular terms are mutually exclusive, reference to “or” indicates either or both possibilities. Thus, for example, reference to “bound or activated” includes bound, activated and both bound and activated.

Another aspect of the present invention describes a mutated PPAR ligand binding domain polypeptide that is a ligand-activated transcription factor. The ligand-activated transcription factor comprises a mutated PPAR ligand binding domain and a transcription factor DNA binding domain. The ligand-activated transcription factor is bound to the DNA response element targeted by the DNA binding domain.

A ligand-activated transcription factor may contain a mutated PPAR ligand binding domain from a particular PPAR subtype along with other PPAR regions from that subtype or may be a chimeric ligand-activated transcription factor. A chimeric ligand-activated transcription factor described herein contains a mutated PPAR ligand binding domain from a particular subtype along with one or more regions from a different nuclear receptor.

Another aspect of the present invention describes a method of making a mutated PPAR ligand binding domain polypeptide. The method involves mutating a PPAR ligand binding domain such that an amino acid present in a wild-type PPAR ligand binding domain that makes a direct interaction with a full agonist is replaced with an amino acid that either makes no interaction, or a substantially different interaction, with the full agonist. If desired additional alterations can be made.

Another aspect of the present invention describes a nucleic acid comprising a nucleotide sequence encoding a mutated PPAR ligand binding domain polypeptide.

Another aspect of the present invention describes a recombinant cell comprising nucleic acid containing a nucleotide sequence encoding a mutated PPAR ligand binding domain polypeptide, wherein the nucleic acid is expressed in the cell. Reference to “expressed” indicates the production of encoded polypeptide.

Another aspect of the present invention describes a method of assaying for a partial PPAR agonist. The method involves measuring the ability of a test compound to bind or activate a mutated PPAR ligand binding domain polypeptide or a transcription factor containing a mutated PPAR ligand binding domain. Measuring can be performed qualitatively or quantitatively.

Other features and advantages of the present invention are apparent from the additional descriptions provided herein including the different examples. The provided examples illustrate different components and methodologies useful in practicing the present invention. The examples do not limit the claimed invention. Based on the present disclosure the skilled artisan can identify and employ other components and methodologies useful for practicing the present invention.

### BRIEF DESCRIPTION OF THE DRAWINGS

FIG. 1 provides the amino acid sequence of a wild type PPARα (SEQ ID NO: 1). Tyr464 is shown in bold. The ligand binding domain is from about amino acid 281 to 468. The DNA binding domain is from about amino acids 102 to 166.

FIG. 2 provides the amino acid sequence of a wild type PPARδ (SEQ ID NO: 2). Tyr437 is shown in bold. The ligand binding domain is from about amino acid 254 to 441. The DNA binding domain is from about amino acids 74 to 138.

FIG. 3 provides the amino acid sequence of a wild type PPARγ (SEQ ID NO: 3). Tyr473 is shown in bold. The ligand binding domain is from about amino acid 203 to 477. The DNA binding domain is from about amino acids 81 to 145.

FIG. 4 illustrates Compound 1 and rosiglitazone-induced transactivation of a PPARγ Tyr473Ala mutant in comparison with wild-type PPARγ response.

FIG. 5 illustrates Compound 1 and rosiglitazone-induced transactivation of a PPARγ Tyr473Phe mutant in comparison with wild-type PPARγ response.

### DETAILED DESCRIPTION OF THE INVENTION

Polypeptides containing mutated PPAR ligand binding domains described herein can be used to facilitate identification and evaluation of partial agonists. Partial agonists have research and therapeutic applications. Research applications include using the partial agonist to study the biological effects of PPAR partial activation or antagonism and to identify important functional groups affecting the ability of a partial agonist to bind to or modulate a PPAR activity.

Therapeutic applications include using those partial agonists having appropriate pharmacological properties such as efficacy and lack of unacceptable toxicity to achieve a beneficial effect in a patient. A partial agonist can be used to provide a beneficial effect of PPAR modulation (e.g., partial activation or antagonism), while producing less side effects than a full agonist.

A “patient” refers to a mammal that can receive a beneficial effect by the administration of a PPAR partial agonist. A patient can be treated prophylactically or therapeutically. Examples of patients include human patients, and non-human patients such as farm animal, pets, and animals that can be used as model systems.

Beneficial effects that can be achieved by modulating one or more PPARs include treatment of one or more of the following: atherosclerosis, dyslipidemia, inflammation, cancer, infertility, hypertension, obesity, and diabetes. (Berger et al., Annu. Rev. Med. 53:409-435, 2002, Berger et al., Diabetes Technology & Therapeutics 4:163-174, 2002, Berger et al., International Publication Number WO 01/30343, published May 3, 2001.)

PPARγ

Using the PPARγ ligand binding domain as a model it was found that alterations can be produced resulting in a mutated ligand binding domain that is selectively bound or activated by a partial agonist. The mutated ligand binding domains illustrated in the Examples infra have a Tyr473Ala or Tyr473Phe substitution.

The full agonist rosiglitazone hydrogen bonds with the PPARγ Tyr473 phenolic hydroxyl, while the partial agonist 1-(p-chlorobenzyl)-5-chloro-3-phenylthiobenzyl-2-yl carboxylic acid (Compound 1) does not hydrogen bond with Tyr473. Replacement of Try473 with an amino acid that does not allow hydrogen bonding to rosiglitazone diminishes an interaction that occurs between rosiglitazone and the AF-2 domain.

Compound 1 and its use as a partial agonist is described by Berger et al., International publication WO 01/30343, published May 3, 2001. Compound 1 has the following structure:

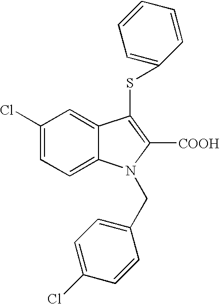

PPARγ ligand binding domain polypeptides in which Tyr473 was replaced with a non-polar amino acid (e.g., alanine or phenylalanine) were found to bind to partial agonist and to activate ligand binding domain activity. Activation of a transcription factor containing a mutated ligand binding domain was at least as good (Tyr473Ala) or significantly better (Tyr473Phe) than that occurring with the wild-type ligand binding domain.

Amino acids involved in agonist and partial agonist binding can be identified using X-ray crystallography. PPARγ ligand binding domain X-ray crystallography data, and techniques for generating such data are illustrated by, for example, Nolte et al., Nature 395:137-143, 1998 and Oberfield et al., Proc. Natl. Acad. Sci. USA 96:6120-6106, 1999.

Amino acids other than Tyr473 can be mutated to diminish binding of a full agonist to the PPARγ AF-2 domain and maintain or facilitate partial agonist binding or activity. The ability of a polypeptide containing a mutated ligand binding domain to be selectively activated or bound by a partial agonist can be evaluated by, for example, measuring the ability of the polypeptide to bind or be activated by a full agonist and partial agonist.

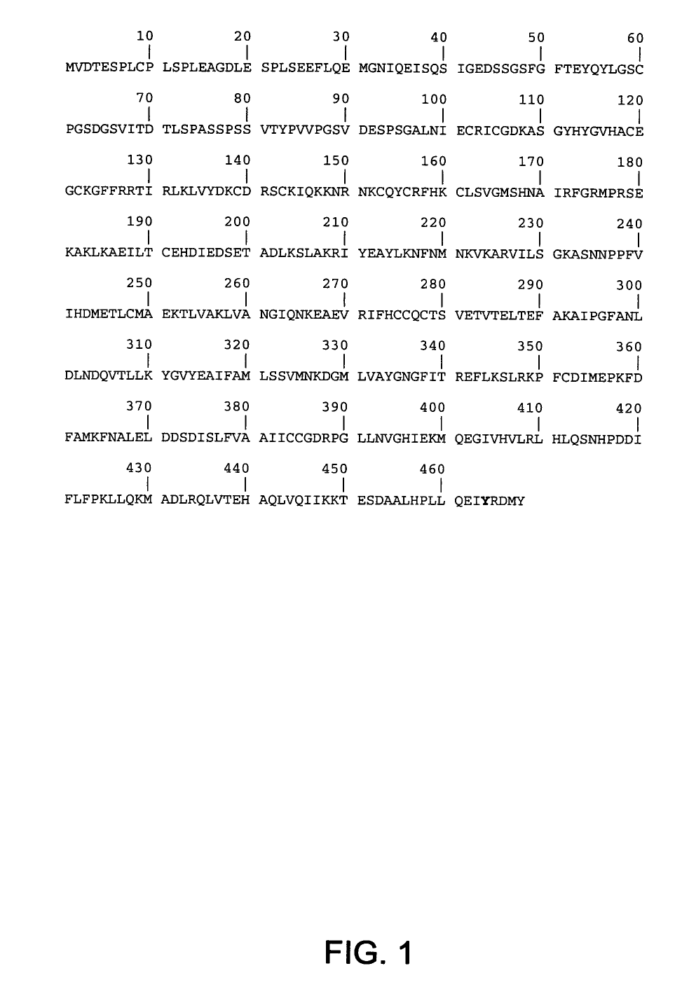

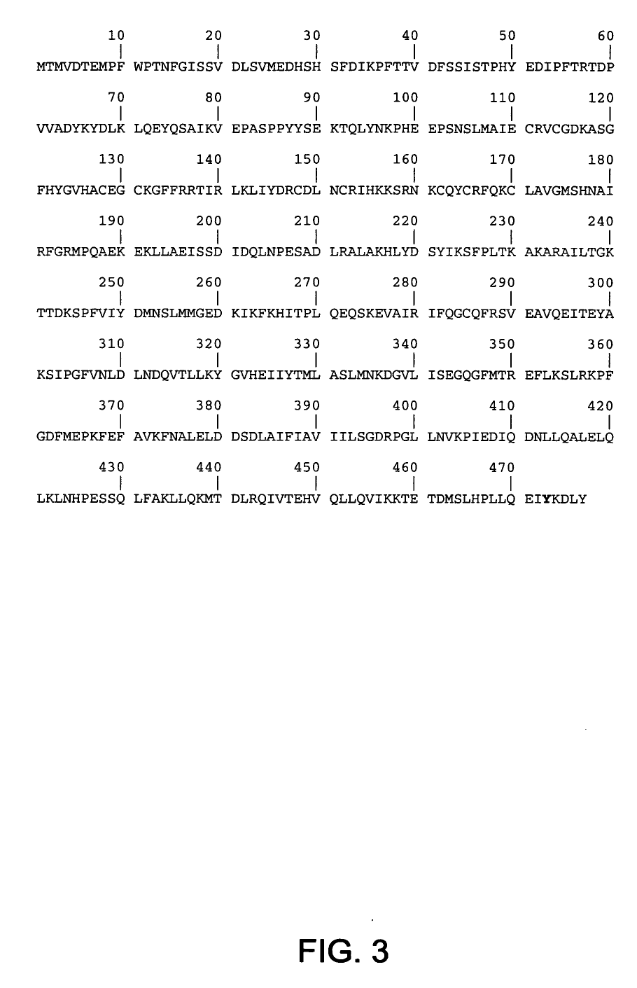

Reference to an amino acid in a particular location such as Tyr473 is with respect to a reference amino acid sequence. Reference amino acid sequences for PPARα, PPRδ, PPARγ are provided by SEQ ID NOs: 1, 2 and 3 (FIG. 1-3). The amino acid numbering for a particular PPAR may differ due to differences in that PPAR that occur in nature or are artificially produced. Naturally occurring differences may be, for example, isoforms and polymorphisms.

The amino acid in a polypeptide corresponding to a referenced amino acid can readily be identified by performing a sequence alignment with a reference sequence. The alignment should be performed to maximize the number of identical amino acids in a region (e.g., 15 or 20 amino acids) containing the amino acid in question.

Replacement of tyrosine 473 with an appropriate amino acid could produce a mutated human PPARγ ligand binding domain with unique properties that can be used to identify the kinds of ligands used to activate the nuclear receptor. In different embodiments, the ligand binding domain is a mutated human PPARγ ligand binding domain, wherein a residue corresponding to tyrosine 473 is selected from a group consisting of:

(a) alanine, valine, leucine, isoleucine, proline, tryptophan, phenylalanine, methionine, histidine, asparagine, and glutamine;

(b) alanine, valine, leucine, isoleucine, proline, tryptophan, phenylalanine, and methionine; or

(c) alanine or phenylalanine.

In another embodiment, the ligand binding domain comprises SEQ ID NO: 4 or a structurally similar sequence. SEQ ID NO: 4 is provided as follows:

QLNPESADLRALAKHLYDSYIKSFPLTKAKARAILTGKTTDKSPFVIYDM
NSLMMGEDKIKFKHITPLQEQSKEVAIRIFQGCQFRSVEAVQEITEYAKS
IPGFVNLDLNDQVTLLKYGVHEIIYTMLASLMNKDGVLISEGQGFMTREF
LKSLRKPFGDFMEPKFEFAVKFNALELDDSDLAIFIAVIILSGDRPGLLN
VKPIEDIQDNLLQALELQLKLNHPESSQLFAKLLQKMTDLRQIVTEHVQL
LQVIKKTETDMSLHPLLQEIXKDLY

wherein X is selected from the group consisting of: alanine, valine, leucine, isoleucine, proline, tryptophan, phenylalanine, methionine, histidine, asparagine, and glutamine. In further embodiments X is selected from the group consisting of: alanine, valine, leucine, isoleucine, proline, tryptophan, phenylalanine, methionine; and X is alanine or phenylalanine.
PPARα and PPARδ

PPARα, PPARδ, and PPARγ contain similar ligand binding domains, where the AF-2 domain contributes to the ligand binding pocket. The AF-2 domain in these receptors provides a ligand-dependent activation domain that participates in the generation of a coactivator binding pocket. (Berger et al., Annu. Rev. Med. 53:409-435, 2002.)

The similarity between different PPAR ligand binding domains and the results obtained using a mutated PPARγ ligand binding domain can be used to guide the design of polypeptides containing a mutated PPARα or PPARδ ligand binding domain. The ability of a polypeptide containing a mutated ligand binding domain to be selectively activated or bound by a partial agonist can be evaluated by, for example, measuring the ability of the polypeptide to bind or be activated by a full agonist and partial agonist.

X-ray crystallography data for PPARα and PPARδ can be generated using techniques well known in the art. X-ray crystallography data for the PPARα ligand binding domain and ligand binding is described by Lambert et al., International Publication Number WO 02/064632, published Aug. 22, 2002. X-ray crystallography data for the PPARδ ligand binding domain and ligand binding is described by Xu et al., Molecular Cell 3:397-403, 1999.

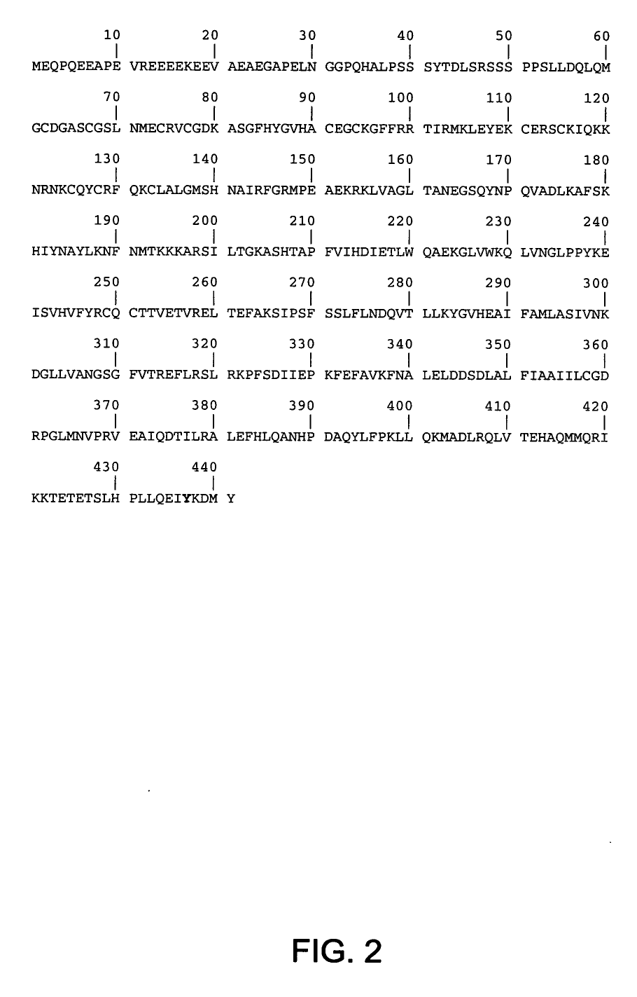

PPARα and PPARδ contain tyrosine residues that function in an analogous manner to Tyr473 in PPARγ. The analogous PPARα tyrosine is in position 464 (FIG. 1). The analogous PPARδ tyrosine is in position 437 (FIG. 2).

Partial agonists for PPARα can be identified, for example, by screening for compounds that activate PPARα where Tyr464 is replaced with an amino acid such as alanine or phenylalanine. Such partial agonists, in addition to the other uses described herein, can be used to obtain or evaluate mutated PPARα ligand binding domain polypeptides and ligand-activated transcription factors.

Similarly, partial agonists for PPARδ can be identified, for example, by screening for compounds that activate PPARδ where Tyr437 is replaced with an amino acid such as alanine or phenylalanine. Such partial agonists, in addition to the other uses described herein, can be used to obtain or evaluate mutated PPARδ ligand binding domain polypeptides and ligand-activated transcription factors.

A mutated human PPARα ligand binding where tyrosine 464 is replaced with an appropriate amino acid could produce a mutated human PPARα ligand binding domain with unique properties that can be used to identify the kinds of ligands used to activate the nuclear receptor. Similarly, a mutated human PPARδ ligand binding where tyrosine 437 is replaced with an appropriate amino acid could produce a mutated human PPARδ ligand binding domain with unique properties that can be used to identify the kinds of ligands used to activate the nuclear receptor.

In different embodiments, the mutated ligand binding domain either is a mutated human PPARα ligand binding domain containing a mutation in a residue corresponding to tyrosine 464, or a mutated human PPARδ ligand binding domain containing a mutation in a residue corresponding to tyrosine 437, wherein the mutation is an amino acid selected from the group consisting of: (a) alanine, valine, leucine, isoleucine, proline, tryptophan, phenylalanine, methionine, histidine, asparagine, and glutamine. In further embodiments, the mutation is either an amino acid selected from the group consisting of alanine, valine, leucine, isoleucine, proline, tryptophan, phenylalanine, and methionine; or is alanine or phenylalanine.

Ligand-Activated Transcription Factor

A ligand-activated transcription factor binds a partial agonist and can modulate gene expression upon partial agonist binding. Based on the interchangeability of different nuclear receptor regions, different types of transcription factors can be produced containing a mutated PPAR ligand binding domain.

Nuclear receptors exhibit a modular structure with different regions corresponding to autonomous functional domains that can be interchanged between related receptors. (Aranda et al., Physiological Reviews 81:1269-1304, 2001.) In different embodiments, a ligand-activated transcription factor is a chimeric receptor containing a mutated PPAR ligand binding domain and one or more regions from another nuclear receptor or other transcription factor (such as GAL4); or is a particular PPAR having a mutated ligand binding domain.

A preferred chimeric receptor described herein is one containing a mutated PPAR ligand binding domain and a DNA binding domain from a different nuclear receptor or other transcription factor (such as GALA). The selection of a particular DNA binding domain is useful in designing a reporter system to measure receptor activity. Examples of DNA binding domains used in PPAR chimeric receptors are the yeast transcription factor Gal4 and the glucocorticoid receptor. (Lehman et al., The Journal of Biological Chemistry 270:12953-12956, 1995, Schmidt et al., Molecular and Cellular Endocrinology 155:51-60, 1999, Berger et al., The Journal of Biological Chemistry 274:6718-6725, 1999.)

Ligand binding domain regions based on a PPAR can be designed starting from known PPAR sequences. Different PPARα, PPARδ, PPARγ sequences include different isoforms and polymorphisms. References providing PPARα sequence information include Sher et al., Biochemistry 32:5598-5604, 1993 (see also SWISS-PROT: QO7869). References providing PPARγ sequence information include Elbrecht et al., Biochem. Biophys. Res. Commun. 224:431-437, 1996 (see also SWISS-PROT: P37231). References providing PPARδ sequence information include Schmidt et al., Mol. Endocrinol. 6:1634-1641, 1993, (see also SWISS-PROT: QO3181).

X-ray crystallography data pointing out the importance of different PPAR amino acid residues to ligand binding and activity can be used to facilitate polypeptide design. References providing examples of X-ray crystallography data and methods of obtaining such data include Lambert et al., International Publication Number WO 02/064632, published Aug. 22, 2002, Xu et al., Molecular Cell 3:397-403, 1999, Nolte et al., Nature 395:137-143, 1998, and Oberfield et al., Proc. Natl. Acad. Sci. USA 96:6120-6106, 1999.

Amino acid alterations can be designed to maintain ligand binding or receptor activity taking into account the structure and property of different amino acids. Depending upon an amino acid side chain (“R” group), amino acids will have different properties such as size, polarity, the ability to hydrogen bond, and hydrophobicity. The effect of different amino acid side chains on properties of an amino acid are well known in the art. (See, for example, Ausubel, Current Protocols in Molecular Biology, John Wiley, 1987-2001, Appendix 1C.)

In exchanging amino acids to maintain activity, the replacement amino acid should have similar properties. For example, substituting valine for leucine, arginine for lysine, and asparagine for glutamine are good candidates for not causing a change in polypeptide functioning.

In exchanging amino acids to diminish an agonist interaction, the replacement amino acid should have a side chain not able to make the same type of interaction as the amino acid being replaced. For example neutral and hydrophobic amino acids (alanine, valine, leucine, isoleucine, proline, tryptophan, phenylalanine, and methionine), are good candidates for diminishing a hydrogen bond interaction. Proline because of its more restricted set of main chain conformations is generally not preferred.

In different embodiments the mutated ligand binding domain, which may be part of a transcription factor, is structurally similar to the ligand binding domain present in SEQ ID NOs: 1, 2, or 3. A structurally similar sequence is at least about 90% identical or similar to a reference sequence. In different embodiments, a structural similar sequence is at least about 95% identical or similar, or at least about 99% identical or similar, to a reference sequence; or differs from the reference sequence by 1, 2, 3, 4, 5, 6, 7, 8, 9, 10, 11, 12, 13, 14, 15, 16, 17, 18, 19, or 20 amino acid alterations.

Percent identity can be calculated by determining the minimum number of amino acid alterations to an amino acid sequence required to arrive at a reference sequence divided by the number of amino acids in the reference sequence multiplying by 100, then subtracting 100 by the obtained number. Amino acid alterations can be any combination of additions, deletions, or substitutions. The amino acid sequence compared to a reference sequence can be part of a larger sequence.

Sequence similarity for polypeptides can be determined by BLAST. (Altschul, et al., 1997. Nucleic Acids Res. 25, 3389-3402, hereby incorporated by reference herein.) In one embodiment sequence similarity is determined using tBLASTn search program with the following parameters: MATRIX:BLOSUM62, PER RESIDUE GAP COST: 11, and Lambda ratio: 1.

In different embodiments, the transcription factor contains a mutated ligand binding domain described herein for PPARα, PPARδ, or PPARγ. In preferred embodiments, the transcription factor consists of the amino acid sequence of SEQ ID NO: 5 or SEQ ID NO: 6. SEQ ID NO: 5 contains a Tyr473Ala alteration, while SEQ ID NO: 6 contains a Tyr473Phe alteration. SEQ ID NOs: 5 and 6 are as follows:

SEQ ID NO: 5:
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVSPGI
RMSHNAIRFGRMPQAEKEKLLAEISSDIDQLNPESADLRALAKHLYDSYI
KSFPLTKAKARAILTGKTTDKSPFVIYDMNSLMMGEDKIKFKHITPLQEQ
SKEVAIRIFQGCQFRSVEAVQEITEYAKSIPGFVNLDLNDQVTLLKYGVH
EIIYTMLASLMNKDGVLISEGQGFMTREFLKSLRKPFGDFMEPKFEFAVK
FNALELDDSDLAIFIAVIILSGDRPGLLNVKPIEDIQDNLLQALELQLKL
NHPESSQLFAKLLQKMTDLRQIVTEHVQLLQVIKKTETDMSLHPLLQEIA
KDLY
SEQ ID NO: 6:
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVSPGI
RMSHNAIRFGRMPQAEKEKLLAEISSDIDQLNPESADLRALAKHLYDSYI
KSFPLTKAKARAILTGKTTDKSPFVIYDMNSLMMGEDKIKFKHITPLQEQ
SKEVAIRIFQGCQFRSVEAVQEITEYAKSIPGFVNLDLNDQVTLLKYGVH
EIIYTMLASLMNKDGVLISEGQGFMTREFLKSLRKPFGDFMEPKFEFAVK
FNALELDDSDLAIFIAVIILSGDRPGLLNVKPIEDIQDNLLQALELQLKL
NHPESSQLFAKLLQKMTDLRQIVTEHVQLLQVIKKTETDMSLHPLLQEIF
KDLY

Polypeptide Production

Polypeptides can be produced using standard techniques including those involving chemical synthesis and those involving biochemical synthesis. Techniques for chemical synthesis of polypeptides are well known in the art. (See e.g., Vincent, in Peptide and Protein Drug Delivery, New York, N.Y., Dekker, 1990.)

Biochemical synthesis techniques for polypeptides are also well known in the art. Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with a particular amino acid sequence and the known degeneracy of the genetic code, a large number of different encoding nucleic acid sequences can be obtained. The degeneracy of the genetic code arises because almost all amino acids are encoded by different combinations of nucleotide triplets or “codons”. Amino acids are encoded by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr-Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid encoding a mutated ligand binding domain can be obtained by producing a nucleic acid using chemical synthesis techniques or by mutating a previously synthesized nucleic acid. Mutating a previously synthesized nucleic acid is facilitated using techniques such as site directed mutagenesis which can be employed to alter a particular nucleotide to obtain a desired codon.

Recombinant Expression

Polypeptides are preferably expressed by recombinant nucleic acid in a suitable host or expression system. Recombinant nucleic acid is nucleic acid that by virtue of its sequence or form does not occur in nature. Possible forms for recombinant nucleic acid include isolation from nucleic acid found in a cell; or a polypeptide encoding region combined with other nucleic acid, which may be present in a host genome or outside of the host genome.

More preferably, expression is achieved in a host cell using an expression vector. An expression vector is a recombinant nucleic acid that includes a region encoding a polypeptide along with regulatory elements for proper transcription and processing. The regulatory elements that may be present include those naturally associated with the polypeptide encoding region and exogenous regulatory elements not naturally associated with the polypeptide coding region.

Exogenous regulatory elements such as an exogenous promoter can be useful for expressing recombinant nucleic acid in a particular host. An exogenous promoter for a polypeptide containing a mutated PPAR ligand binding domain is a promoter that is not naturally associated with PPAR encoding nucleic acid.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

To enhance expression in a particular host it may be useful to modify a particular encoding sequence to take into account codon usage of the host. Codon usage of different organisms are well known in the art. (See, Ausubel, Current Protocols in Molecular Biology, John Wiley, 1987-1998, Supplement 33 Appendix 1C.)

Expression vectors may be introduced into host cells using standard techniques. Examples of such techniques include transformation, transfection, lipofection, protoplast fusion, and electroporation.

Nucleic acid encoding a polypeptide can be expressed in a cell without the use of an expression vector. For example, mRNA can be translated in various cell-free systems such as wheat germ extracts and reticulocyte extracts, as well as in cell based systems, such as frog oocytes. Introduction of mRNA into cell based systems can be achieved, for example, by microinjection.

PPAR assays can be performed using a host expressing a mutated ligand binding domain polypeptide, and can be performed using a mutated ligand binding domain polypeptide purified from a host or expression system. Preferably, assays are performed using a recombinant cell.

A recombinant cell encoding a mutated PPAR ligand binding domain polypeptide is a cell that is modified to contain nucleic acid encoding the polypeptide. The modification can be by different methods, such as introduction of an expression vector and mutation of the host genome.

PPAR Assays Formats

Polypeptides containing a mutated PPAR ligand binding domain can be employed to evaluate and select for partial agonists. A variety of different assay formats can be employed including ligand binding assays, assays measuring coactivator affinity, and assay measuring transcription factor activity. Examples of different assay formats include:

1) Measuring ligand binding using a scintillation proximity assay format (e.g., Elbrecht et al., The Journal of Biological Chemistry 12:7913-7922, 1999);

2) Measuring nuclear receptor affinity for cofactors using fluorescence resonance energy transfer (e.g., Zhou et al., Molecular Endocrinology 12:1594-1604, 1998); and

3) Measuring transcription factor activity (e.g., Example Section infra., Lehman et al., The Journal of Biological Chemistry 270:12953-12956, 1995, Schmidt et al., Molecular and Cellular Endocrinology 155:51-60, 1999, Berger et al., The Journal of Biological Chemistry 274:6718-6725, 1999.)

Full and partial agonists can be discriminated, for example, by running two simultaneous transactivation assays one involving the wild-type receptor (native or chimera) and the other involving the mutated receptor. Ligands having severely diminished activity in the mutant assay versus wild-type are classified as full agonists. Ligands that exhibit the same activity or enhanced activity in the mutant assay versus wild-type can be classified as partial agonists.

### EXAMPLES

Examples are provided below further illustrating different features of the present invention. The examples also illustrate useful methodology for practicing the invention. These examples do not limit the claimed invention.

### Example 1

### Mutated Ligand Binding Domain Construction

Mutated PPARγ ligand binding domain polypeptides were generated by site directed mutagenesis of encoding nucleic acid, followed by nucleic acid expression. The starting construct for mutagenesis was pcDNA3-hPPARγ/GAL4. pcDNA3-hPPARγ/GAL4 is a chimeric transcription factor containing a human hPPARγ ligand binding domain and a yeast GAL4 transcription factor DNA binding domain.

pcDNA3-hPPARγ/GAL4 was prepared by inserting the yeast GAL4 transcription factor DNA binding domain adjacent to the ligand binding domain of human PPARγ within the mammalian expression vector pcDNA3.1(+). Construction was achieved using techniques described by Elbrecht et al. J. Biol. Chem. 274:7913-7922, 1999.

Starting with pcDNA3-hPPARγ/GAL4, the Tyr473 residue of human PPARγ was mutated to Ala or Phe by utilizing the Quikchange Site-Directed Mutagenesis Kit according to the protocol of the manufacturer (Stratagene, La Jolla, Calif.). The Tyr473Ala mutation was made using the forward oligonucleotide 5′-GCTCCTGCAGGAGATCGCCAAGGACTTGTACTAG-3′ (SEQ ID NO: 9) and the reverse oligonucleotide 5′-CTAGTACAAGTCCTTGGCGATCTCCTGCAGGAGC-3′ (SEQ ID NO: 10). The Tyr473Phe mutation was made using the forward oligonucleotide 5′-GCTCCTGCAGGAGATCTTCAAGGACTTGTACTAG-3′ (SEQ ID NO: 11) and the reverse oligonucleotide 5′-CTAGTACAAGTCCTTGAAGATCTCCTGCAG GAGC-3′ (SEQ ID NO: 12).

The mutated constructs containing a PPARγ ligand binding alteration in Tyr473 were designated pcDNA3-PPARγ(473Ala)/GAL4, or pcDNA3-PPARγ(473Phe)/GAL4. The nucleic acid sequence encoding the GAL4/PPARγ (473 Ala) construct is provided by SEQ ID NO: 7. The nucleic acid sequence encoding the GAL4/PPARγ (473 Phe) construct is provided by SEQ ID NO: 8.

### Example 2

### Transactivation Assay

A transactivation assay was performed to evaluate mutated PPAR PPARγ ligand binding domains. The transcription assay employed the transcription factors described in Example 1 and a reporter plasmid. Expression of the reporter plasmid is induced by transcription factor activation.

The employed reporter plasmid for the GAL4 chimeric receptors (pUAS(5×)-tk-luc) contains five repeats of the GAL4 response element (UAS) upstream of a minimal thymidine kinase promoter that is adjacent to the luciferase gene. (Berger et al., J. Biol. Chem. 274:6718-6725, 1999.) A control vector, pCMV-lacZ, contains the CMV promoter adjacent to the galactosidase Z gene. (Berger et al., J. Biol. Chem. 274:6718-6725, 1999.)

Rosiglitazone ((+/−)-5-(4-(2-(methyl-2-pyridinylamino)ethoxy)phenyl)methyl)-2,4-thiazolidinedione) and Compound 1 were evaluated. Cell culture reagents were obtained from Gibco (Gaithersburg, Md.). Unless otherwise noted, all other reagents were obtained from Sigma Chemicals (St. Louis, Mo.).

COS-1 cells were cultured and transactivation assays were performed using the expression vectors pcDNA3-PPARγ/GAL4, pcDNA3-PPARγ(473Ala)/GAL4, or pcDNA3-PPARγ(473Phe)/GAL4 using techniques described by Berger et al., J. Biol. Chem. 274:6718-6725, 1999. Briefly, cells were transfected with a transcription factor expression vector, pUAS(5×)-tk-luc reporter vector and pCMV-lacZ as an internal control for transactivation efficiency using Lipofectamine (Invitrogen, Carlsburg, Calif.). After a 48 hour exposure to compounds, cell lysates were produced, and luciferase and β-galactosidase activity in cell extracts was determined. (Berger et al., J. Biol. Chem. 274:6718-6725, 1999.)

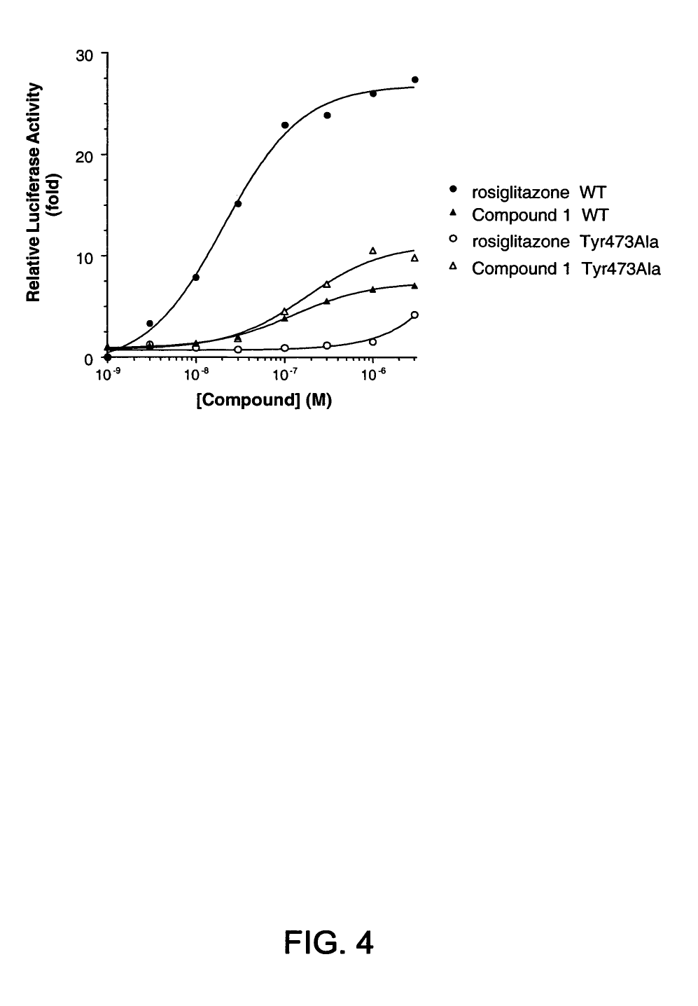

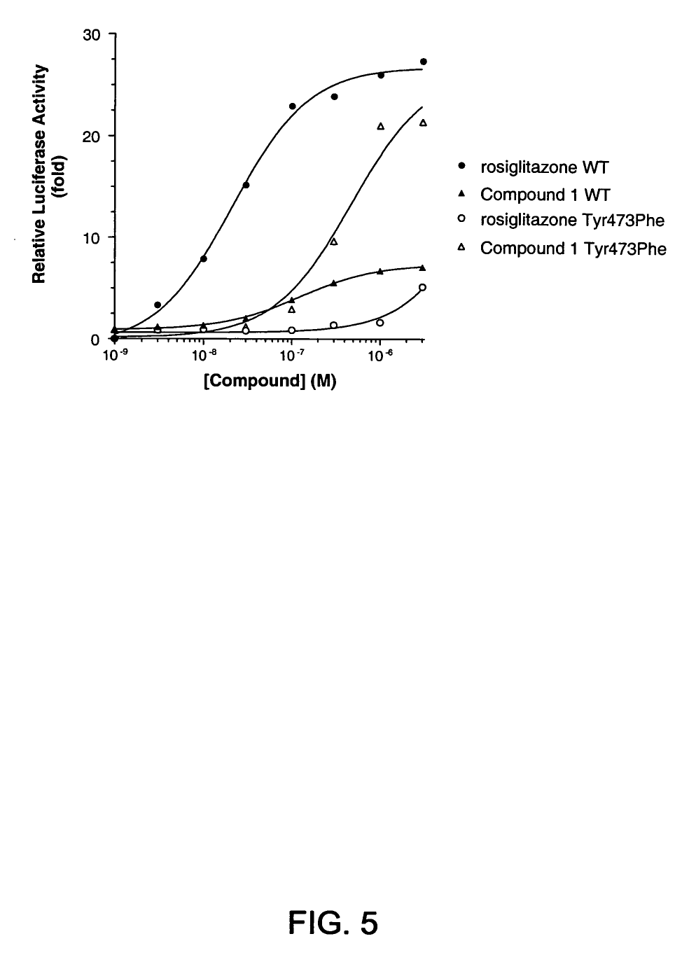

The PPARγ full agonist rosiglitazone showed a dramatic diminution in potency in activating the PPARγ Tyr473Ala mutant in comparison with wild-type PPARγ (FIG. 4). In contrast, the potency of Compound 1 in activating the PPARγ Tyr473Ala mutant remained essentially unchanged while its efficacy (maximal response) was augmented in comparison with wild-type PPARγ (FIG. 4). The potency of rosiglitazone in activating the PPARγ Tyr473Phe mutant was also greatly reduced in comparison with wild-type PPARγ (FIG. 5). The potency of Compound 1 in activating the PPARγ Tyr473Phe remained similar while its efficacy was significantly augmented in comparison with wild-type PPARγ (FIG. 5).

Other embodiments are within the following claims. While several embodiments have been shown and described, various modifications may be made without departing from the spirit and scope of the present invention.

1. A mutated peroxisome proliferator-activated receptor (PPAR) ligand binding domain polypeptide comprising the amino acid sequence of a mutated PPAR ligand binding domain, wherein said mutated PPAR ligand binding domain is
(a) bound by a partial PPAR agonist; and
(b) bound or activated by a full PPAR agonist to a lesser extent than the wild-type receptor.

2. The mutated PPAR ligand binding domain polypeptide of claim 1, wherein said mutated PPAR ligand binding domain selectively binds said partial agonist.

3. The mutated PPAR ligand binding domain polypeptide of claim 1, wherein said mutated PPAR ligand binding domain polypeptide is selectively activated by said partial agonist.

4. The mutated PPAR ligand binding domain polypeptide of claim 1, wherein said mutated ligand bind domain is either:
a mutated human PPARα ligand binding domain, wherein a residue corresponding to tyrosine 464 is selected from the group consisting of: alanine, valine, leucine, isoleucine, proline, tryptophan, phenylalanine, methionine, histidine, asparagine, and glutamine;
a mutated human PPARδ ligand binding domain, wherein a residue corresponding to tyrosine 437 is selected from the group consisting of: alanine, valine, leucine, isoleucine, proline, tryptophan, phenylalanine, methionine, histidine, asparagine, and glutamine, or
a mutated human PPARγ ligand binding domain, wherein a residue corresponding to tyrosine 473 is selected from the group consisting of: alanine, valine, leucine, isoleucine, proline, tryptophan, phenylalanine, methionine, histidine, asparagine, and glutamine.

5. The mutated PPAR ligand binding domain polypeptide of claim 1, where said polypeptide comprises the amino acid sequence of SEQ ID NO: 4:
QLNPESADLRALAKHLYDSYIKSFPLTKAKARAILTGKTTDKSPFVIYDM
NSLMMGEDKIKFKHITPLQEQSKEVAIRIFQGCQFRSVEAVQEITEYAKS
IPGFVNLDLNDQVTLLKYGVHEIIYTMLASLMNKDGVLISEGQGFMTREF
LKSLRKPFGDFMEPKFEFAVKFNALELDDSDLAIFIAVIILSGDRPGLLN
VKPIEDIQDNLLQALELQLKLNHPESSQLFAKLLQKMTDLRQIVTEHVQL
LQVIKKTETDMSLHPLLQEIXKDLY
wherein X is selected from the group consisting of: alanine, valine, leucine, isoleucine, proline, tryptophan, phenylalanine, methionine, histidine, asparagine, and glutamine.

6. The mutated PPAR ligand binding domain polypeptide of claim 5, wherein X is phenylalanine or alanine.

7. A ligand-activated transcription factor comprising the mutated PPAR ligand binding domain of claim 1 and a DNA binding domain.

8. The ligand-activated transcription factor of claim 7, wherein said transcription factor can be selectively activated by partial agonist binding.

9. The ligand-activated transcription factor of claim 8, wherein said mutated ligand bind domain is either:
a mutated human PPARα ligand binding domain, wherein a residue corresponding to tyrosine 464 is selected from the group consisting of: alanine, valine, leucine, isoleucine, proline, tryptophan, phenylalanine, methionine, histidine, asparagine, and glutamine;
a mutated human PPARδ ligand binding domain, wherein a residue corresponding to tyrosine 437 is selected from the group consisting of: alanine, valine, leucine, isoleucine, proline, tryptophan, phenylalanine, methionine, histidine, asparagine, and glutamine, or
a mutated human PPARγ ligand binding domain, wherein a residue corresponding to tyrosine 473 is selected from the group consisting of: alanine, valine, leucine, isoleucine, proline, tryptophan, phenylalanine, methionine, histidine, asparagine, and glutamine.

10. The ligand-activated transcription factor of claim 7, where said mutated ligand binding domain consists of the amino acid sequence of SEQ ID NO: 4:
QLNPESADLRALAKHLYDSYIKSFPLTKAKARAILTGKTTDKSPFVIYDM
NSLMMGEDKIKFKHITPLQEQSKEVAIRIFQGCQFRSVEAVQEITEYAK
SIPGFVNLDLNDQVTLLKYGVHEIIYTMLASLMNKDGVLISEGQGFMTRE
FLKSLRKPFGDFMEPKFEFAVKFNALELDDSDLAIFIAVIILSGDRPGLL
NVKPIEDIQDNLLQALELQLKLNHPESSQLFAKLLQKMTDLRQIVTEHVQ
LLQVIKKTETDMSLHPLLQEIXKDLY
wherein X is selected from the group consisting of: alanine, valine, leucine, isoleucine, proline, tryptophan, phenylalanine, methionine, histidine, asparagine, and glutamine.

11. The ligand-activated transcription factor of claim 10, wherein X is phenylalanine or alanine.

12. The ligand-activated transcription factor of claim 11, wherein said transcription factor is a chimeric receptor.

13. The ligand-activated transcription factor of claim 12, wherein said transcription factor consists of the amino acid sequence of SEQ ID NO: 5 or SEQ ID NO: 6.

14. A method of making a mutated PPAR ligand binding domain polypeptide comprising the step of mutating a PPAR ligand binding domain such that an amino acid present in a wild-type PPAR ligand binding domain that makes a direct interaction with a full agonist either makes no interaction, or a substantially different interaction, with said full agonist.

15. The method of claim 14, wherein said mutating produces said mutated PPAR ligand binding domain polypeptide such that said mutated PPAR ligand binding is selectively bound or activated by a partial PPAR agonist.

16. The method of claim 15, wherein said mutating comprises changing an amino acid that makes a direct interaction with a full agonist into an amino acid that either makes no interaction, or a substantially different interaction, with said full agonist.

17. The method of claim 16, wherein said PPAR ligand binding domain that is mutated comprises SEQ ID NO: 3:
QLNPESADLRALAKHLYDSYIKSFPLTKAKARAILTGKTTDKSPFVIYDM
NSLMMGEDKIKFKHITPLQEQSKEVAIRIFQGCQFRSVEAVQEITEYAKS
IPGFVNLDLNDQVTLLKYGVHEIIYTMLASLMNKDGVLISEGQGFMTREF
LKSLRKPFGDFMEPKFEFAVKFNALELDDSDLAIFIAVIILSGDRPGLLN
VKPIEDIQDNLLQALELQLKLNHPESSQLFAKLLQKMTDLRQIVTEHVQL
LQVIKKTETDMSLHPLLQEIYKDLY.

18. A nucleic acid comprising a nucleotide sequence encoding the polypeptide of claim 1.

19-21. (canceled)

22. A method of assaying for a partial PPAR agonist comprising the step of measuring the ability of a test compound to bind to or activate the polypeptide of claim 1.

23. A nucleic acid comprising a nucleotide sequence encoding the transcription factor of claim 7.


SEQUENCE LISTING (12 sequences)

SEQ ID NO: 1 - protein, 468 aa, Human
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 2 - protein, 441 aa, Human
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 3 - protein, 477 aa, Human
MTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 4 - protein, 275 aa, Artificial Sequence
  mutated PPAR ligand binding domain
QLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIXKDLY

SEQ ID NO: 5 - protein, 454 aa, Artificial Sequence
  transcription factor containing a mutated PPAR ligand binding domain
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPGIRMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIAKDLY

SEQ ID NO: 6 - protein, 454 aa, Artificial Sequence
  transcription factor containing a mutated PPAR ligand binding domain
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPGIRMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIFKDLY

SEQ ID NO: 7 - DNA, 1365 bp, Artificial Sequence
  nucleic acid sequence encoding GAL4/PPARg (473 Ala)
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggggatccggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatcgccaaggacttgtactag

SEQ ID NO: 8 - DNA, 1365 bp, Artificial Sequence
  nucleic acid sequence encoding GAL4/PPARg (473 Phe)
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggggatccggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatcttcaaggacttgtactag

SEQ ID NO: 9 - DNA, 34 bp, Artificial Sequence
  oligonucleotide primer
gctcctgcaggagatcgccaaggacttgtactag

SEQ ID NO: 10 - DNA, 34 bp, Artificial Sequence
  oligonucleotide primer
ctagtacaagtccttggcgatctcctgcaggagc

SEQ ID NO: 11 - DNA, 34 bp, Artificial Sequence
  oligonucleotide primer
gctcctgcaggagatcttcaaggacttgtactag

SEQ ID NO: 12 - DNA, 34 bp, Artificial Sequence
  oligonucleotide primer
ctagtacaagtccttgaagatctcctgcaggagc